(19) United States
(12) Patent Application Publication
(10) Pub. No.: US 2014/0303733 A1
(43) Pub. Date: Oct. 9, 2014

(54) BIOLOGICALLY ACTIVE INSERT FOR USE WITH SPINAL FUSION IMPLANTS

(71) Applicant: Timothy T. DAVIS, Santa Monica, CA (US)

(72) Inventor: Timothy T. DAVIS, Santa Monica, CA (US)

(21) Appl. No.: 14/210,782

(22) Filed: Mar. 14, 2014

Related U.S. Application Data
(60) Provisional application No. 61/793,386, filed on Mar. 15, 2013.

(51) Int. Cl. A61F 2/44

(52) U.S. Cl. CPC A61F 2/4455
USPC 623/17.16

(57) ABSTRACT

Biologically active components for use with fusion promoting implantable devices are provided. These components are configured as inserts that work in cooperation with the implantable devices, such as for example spinal fusion implants, to facilitate fusion in a time controlled and directional manner.

### CROSS-REFERENCE TO RELATED APPLICATIONS

This application claims priority to U.S. Provisional No. 61/793,386 filed Feb. 15, 2013 and entitled “BIOLOGICALLY ACTIVE INSERT FOR USE WITH SPINAL FUSION IMPLANTS,” the contents of which are incorporated by reference in their entirety.

### FIELD

The present invention relates to implantable devices for spinal fusion, and more particularly to a biologically active insert configured for use with spinal fusion implants. The biologically active insert works in cooperation with spinal fusion implants to facilitate fusion in a time controlled and directional manner.

### BACKGROUND

Spinal fusion surgery joins or fuses two vertebral bodies together. Spinal fusion surgery is needed to relieve pain, correct alignments of the spine, or repair loss of bone. For example, spinal fusion surgery can be used to treat a herniated vertebral disc in the spinal column.

The two most common techniques utilized in spinal fusion surgery are spinal implants and bone grafts. Spinal implants serve as a mechanical support to secure vertebrae together until new bone grows in between the two vertebral bodies. Bone grafts, taken from other sources of bone in the patient's body, make a bridge between the adjacent vertebrae. In either technique, new bone growth must occur to fix the patient's symptoms and conditions. Therefore, a successful method to allow and promote new bone growth is necessary.

Currently, surgeons utilize various forms of implants to allow and promote new bone growth. Implantable discs and screws can have various shapes, sizes, grooves, and slots. Biological materials and compounds, such as chemotherapeutic or palliative drugs, salts, and bone stimulants, can either coat the implant itself or be deposited into the grooves and slots. Over time, these drugs and stimulants gradually infuse into the surrounding areas for various purposes. For example, they can prevent blood clots, infections, and pain, or promote healing and stabilization. A common growth factor, bone morphogenetic protein (BMP), when used with implants, creates spinal fusion comparable to a patient's own bone and eliminates the need for bone graft.

As mentioned above, the goals of spinal fusion surgery are to initially fuse vertebral bodies together (i.e., stabilize), and then allow and promote new bone growth (i.e., strengthen). However, when an implant is initially inserted, it may be difficult for the implant to immediately adhere to the vertebral body. Also, there can be overzealous bone formation because of the presence of a growth factor, which could cause unintended fusion of adjacent spinal segments and compression of nerves. Accordingly, it would be desirable to provide methods and instruments for improving the initial fuse time between the implant and vertebral body and giving directionality to new bone growth.

### SUMMARY

The present disclosure provides embodiments for a biologically active insert that works in cooperation with spinal fusion implants to facilitate fusion in a time controlled and directional manner. The inserts are customized to fit neatly within the cavities of most spinal fusion implants currently available.

In one embodiment, a biocompatible, bioresorbable insert is provided. The insert may comprise a polymeric shell containing a biologically active agent for facilitating bone fusion. The polymeric shell may have varying thickness to provide directional and time-release control over the biologically active agent. The insert may be configured to seat flush within a central opening of a spinal fusion implant.

The polymeric shell may comprise a biocompatible, bioabsorbable polymer or film-forming agent such as polycaprolactones (PCL), polyglycolic acid (PGA), poly-L-Lactic acid (PL-LA), polysulfones, polyolefins, polyvinyl alcohol (PVA), polyalkenoics, polyacrylic acids (PAA), PEG, PLGA, or polyesters. The biologically active agent may comprise a bone morphogenic protein, a peptide, a bone growth factor, a platelet derived growth factor, a vascular endothelial growth factor, an insulin derived growth factor, a keratinocyte derived growth factor, a fibroblast derived growth factor, a stem cell, bone marrow, or platelet rich plasma. The spinal fusion implant of a type such as a posterior lumbar interbody fusion (PLIF) implant, transforaminal lumbar interbody fusion (TLIF) implant, anterior lumbar interbody fusion (ALIF) implant, or extreme lateral interbody fusion (XLIF) implant.

In some embodiments, the polymeric shell can comprise a superior wall, inferior wall, and sidewalls extending in between. One of the superior or inferior walls may be thinner than the sidewalls. The biologically active agent may be uniformly distributed throughout the insert, or it may be non-uniformly distributed throughout the insert. Further, the biologically active agent may be contained within the shell, and may be uniformly distributed throughout the shell of the insert, or it may be non-uniformly distributed throughout the shell of the insert.

In another embodiment, a spinal fusion implant system is provided. The system may include a spinal fusion implant having a superior surface, an inferior surface, sidewalls extending therebetween, and an opening extending from the superior surface through the inferior surface to accommodate bone growth therethrough. In addition, the system may comprise a biologically active component configured for insertion into the opening of the spinal fusion implant, the component including a polymeric shell containing a biologically active agent for facilitating bone fusion, the polymeric shell having varying thickness to provide directional and time-released control of the biologically active agent, wherein the component has a size and shape that complements the opening and is configured to seat flush within the opening of the spinal fusion implant.

The polymeric shell may comprise a biocompatible, bioabsorbable polymer or film-forming agent such as polycaprolactones (PCL), polyglycolic acid (PGA), poly-L-Lactic acid (PL-LA), polysulfones, polyolefins, polyvinyl alcohol (PVA), polyalkenoics, polyacrylic acids (PAA), PEG, PLGA, or polyesters. The biologically active agent may comprise a bone morphogenic protein, a peptide, a bone growth factor, a platelet derived growth factor, a vascular endothelial growth factor, an insulin derived growth factor, a keratinocyte derived growth factor, a fibroblast derived growth factor, a stem cell, bone marrow, or platelet rich plasma. The spinal fusion implant of a type such as a posterior lumbar interbody fusion (PLIF) implant, transforaminal lumbar interbody fusion (TLIF) implant, anterior lumbar interbody fusion (ALIF) implant, or extreme lateral interbody fusion (XLIF) implant.

In some embodiments, the polymeric shell can comprise a superior wall, inferior wall, and sidewalls extending in between. One of the superior or inferior walls may be thinner than the sidewalls. The biologically active agent may be uniformly distributed throughout the component, or it may be non-uniformly distributed throughout the component. Further, the biologically active agent may be contained within the shell, and may be uniformly distributed throughout the shell of the component, or it may be non-uniformly distributed throughout the shell of the component.

The opening for receiving the insert or component may be a central opening of the spinal fusion implant. However, in other embodiments, the spinal fusion implant can include a plurality of openings, and the system can provide a plurality of components for insertion into the plurality of openings. The concentration of biological agent may differ within one opening to another opening of the spinal fusion implant.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1A shows an exploded view of a spinal fusion implant system comprising a posterior lumbar interbody fusion (PLIF) implant with an exemplary biologically active insert of the present disclosure.

FIG. 1B shows a perspective view of a fully assembled spinal fusion implant system of FIG. 1A.

FIG. 2A shows an exploded view of a spinal fusion implant system comprising an extreme lateral interbody fusion (XLIF) implant with an exemplary biologically active insert of the present disclosure.

FIG. 2B shows a perspective view of a fully assembled spinal fusion implant system of FIG. 2A.

FIG. 3A shows an exploded view of a spinal fusion implant system comprising an anterior lumbar interbody fusion (ALIF) implant with an exemplary biologically active insert of the present disclosure.

FIG. 3B shows a perspective view of a fully assembled spinal fusion implant system of FIG. 3A.

FIG. 4 shows a cross-sectional view of the biologically active insert of FIG. 3A.

FIG. 5 shows a cross-sectional cutaway view of the biologically active insert of FIG. 3B in situ.

FIGS. 6A-6C show the various stages of biological activity of the biologically active insert of FIG. 5 in situ over time.

FIG. 7 shows an exploded view of a spinal fusion implant system comprising an interbody implant in use with exemplary biologically active inserts of the present disclosure.

The foregoing and other features of the present disclosure will become apparent to one skilled in the art to which the present disclosure relates upon consideration of the following description of exemplary embodiments with reference to the accompanying drawings.

### DESCRIPTION OF THE EMBODIMENTS

The embodiments provide a biologically active component for use with fusion promoting implantable devices. These components are configured as inserts that work in cooperation with the implantable devices, such as for example spinal fusion implants, to facilitate fusion in a time controlled and directional manner. The inserts are intended to be customized to fit currently existing devices such as spinal fusion implants, including without limitation, posterior lumbar interbody fusion (PLIF) implants, transforaminal lumbar interbody fusion (TLIF) implants, anterior lumbar interbody fusion (ALIF) implants, and extreme lateral interbody fusion (XLIF) implants. In some cases, the biologically active inserts of the present disclosure may be use alone, without a fusion cage or implant, depending the location, size and geometry of the area to be fused. In addition, the insert only releases the agent at the superior and inferior surfaces where contact with bleeding bone is optimal.

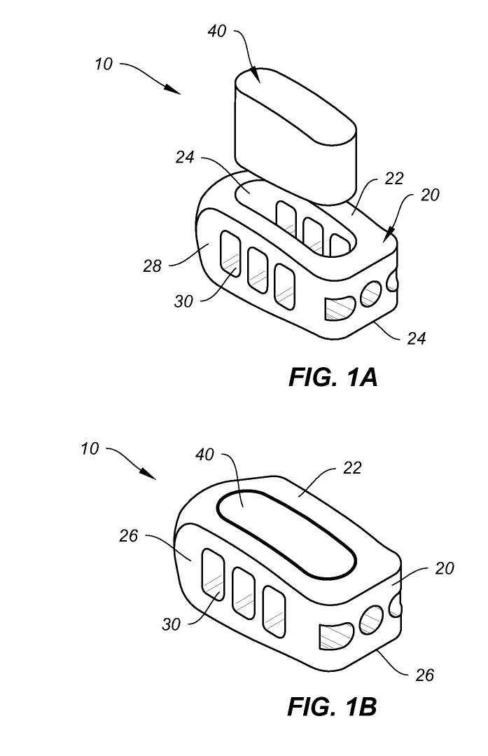

Turning now to the drawings, FIG. 1A shows an exploded view of a spinal fusion implant system 10 comprising an exemplary embodiment of a known posterior lumbar interbody fusion (PLIF) implant 20 with an exemplary biologically active insert 40 of the present disclosure. The insert 40 is configured to nest securely within the central opening 24 of the implant 20. As shown in FIG. 1B, the insert 40 may be shaped and sized to complement the geometry of the central opening 24 such that, when assembled, the insert 40 sits flush within the implant 20.

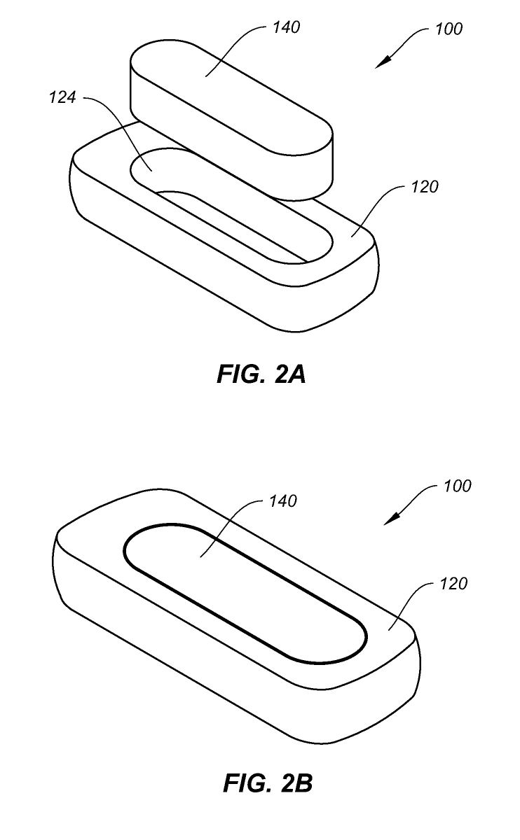

It is contemplated that the inserts of the present disclosure may have any size, shape or geometry that is complementary to currently available fusion promoting implants. For instance, FIG. 2A shows an exploded view of a spinal fusion implant system 100 comprising an exemplary embodiment of a known extreme lateral interbody fusion (XLIF) implant 120 with an exemplary biologically active insert 140 of the present disclosure. FIG. 2B shows a fully assembled spinal fusion implant system 100 in which the insert 140 of the present disclosure nests closely within the central opening 124 of the implant 120.

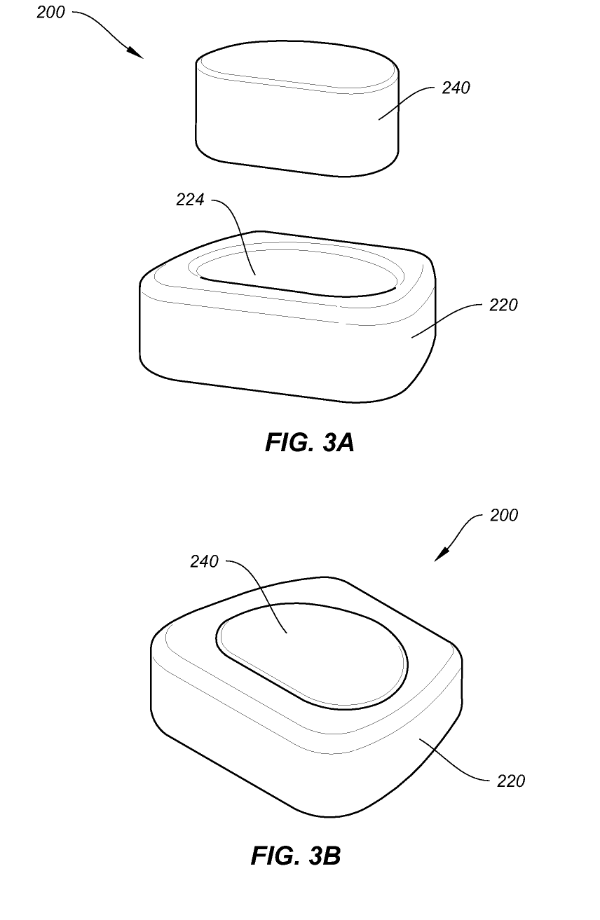

In another example, FIG. 3A shows an exploded view of a spinal fusion implant system 200 comprising an exemplary embodiment of a known anterior lumbar interbody fusion (ALIF) implant 220 with an exemplary biologically active insert 240 of the present disclosure. FIG. 3B shows a fully assembled spinal fusion implant system 200 in which the insert 240 of the present disclosure nests closely within the central opening 224 of the implant 220.

The biologically active insert 240 may be formed of a biocompatible, bioresorbable material. For example, biocompatible, bioabsorbable polymer or film-forming agents such as polycaprolactones (PCL), polyglycolic acid (PGA), poly-L-Lactic acid (PL-LA), polysulfones, polyolefins, polyvinyl alcohol (PVA), polyalkenoics, polyacrylic acids (PAA), PEG, PLGA, polyesters and the like are suitable materials for the insert 240. Other suitable materials also include artificial polymers selected from poly(anhydrides), poly(hydroxy acids), polyesters, poly(orthoesters), polycarbonates, poly(propylene fumerates), poly(caprolactones), polyamides, polyamino acids, polyacetals, polylactides, polyglycolides, polysulfones, poly(dioxanones), polyhydroxybutyrates, polyhydroxyvalyrates, poly(vinyl pyrrolidones), biodegradable polycyanoacrylates, biodegradable polyurethanes, polysaccharides, tyrosine-based polymers, poly(methyl vinyl ether), poly(maleic anhydride), poly(glyconates), polyphosphazines, poly(esteramides), polyketals, poly(orthocarbonates), poly(maleic acid), poly(alkylene oxalates), poly(alkylene succinates), poly(pyrrole), poly(aniline), poly(thiophene), polystyrene, non-biodegradable polyurethanes, polyureas, poly(ethylene vinyl acetate), polypropylene, polymethacrylate, polyethylene, poly(ethylene oxide), and co-polymers, adducts, and mixtures thereof.

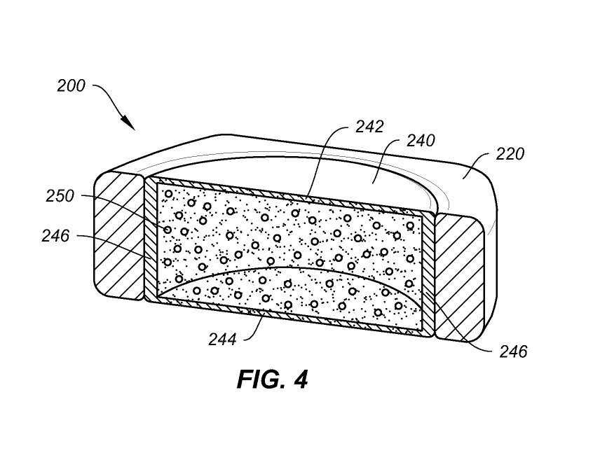

As shown in FIG. 4, the insert 240 may serve as a delivery device or vehicle for a biologically active agent 250, preferably an agent that enhances or facilitates fusion. Such an agent could be bone morphogenic protein (BMP). However, other biological agents may be substituted for, or supplement, the BMP, including a peptide, a bone growth factor, a platelet derived growth factor, a vascular endothelial growth factor, an insulin derived growth factor, a keratinocyte derived growth factor, a fibroblast derived growth factor, a stem cell, bone marrow, or platelet rich plasma, to name a few exemplary agents.

The biologically active inserts of the present disclosure may provide time-released control as well as directional control of the biological agents to be delivered. In order to control the timing and directionality of the release, the biologically active insert can be configured with varying wall thicknesses. As the cross-sectional view of FIG. 4 illustrates, in one exemplary embodiment, the superior and inferior surfaces 242, 244 of the insert may be thinner than the sidewalls 246 of the insert, and thus absorb faster. Upon implantation, as the superior and inferior surfaces 242, 244 resorb, the biological agent 250 within the insert 240 will be released from these sides first. If a large quantity of agent 250 is desired initially, these superior and inferior walls 242, 244 may be very thin. Accordingly, the insert 240 holds the biologically active agent in place without random dispersion prior to closure of the wound or surgical site. In addition, as configured the insert 240 only releases the agent 250 at the superior and inferior surfaces where contact with bleeding bone is optimal.

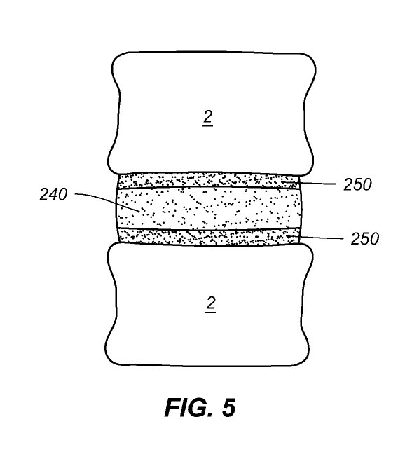

In another embodiment, the insert 240 may be configured such that the biological agent 250 is more concentrated near the superior 242 and inferior 244 surfaces of the insert 240. This could be achieved by providing a multi-layered insert in which the concentration of biological agent 250 can differ from one layer to the next, such as represented in FIG. 5. Another way to concentrate the biological agent 250 is to encase the agent 250 within another bioresorbable coating or layer. The biological agents 250 near the superior and inferior surfaces 242, 244 of the insert 240 could then be thinly coated, or provided without a coating, so that they react faster than the remaining agents 250.

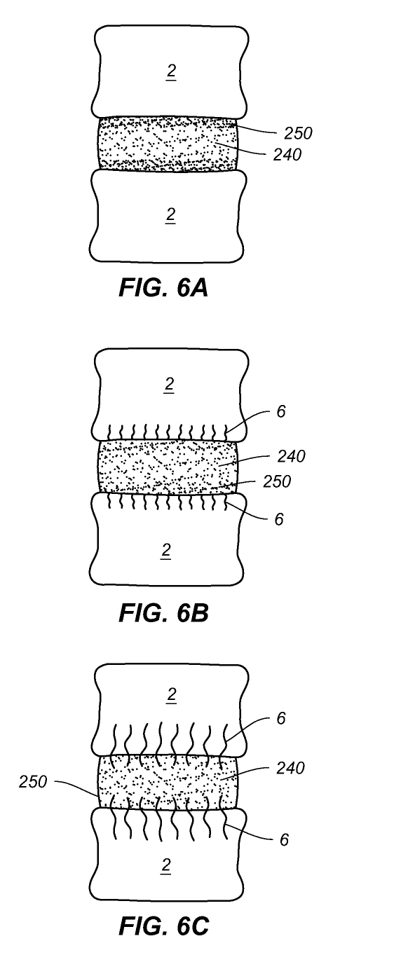

As mentioned, one of the advantages of the biologically active inserts of the present disclosure is the ability to have directional control. As illustrated in FIGS. 6A-6C, the inserts 240 may be configured so as to provide a large burst of bioactivity via the agents 250 near the endplates of the vertebrae 2. Rather than uniform dispersion of the agents, the inserts 240 focus and guide the new growth initially where it is most needed, which is at the endplates of the vertebrae 2. Later, after good bone purchase or new growth has been established, the agents 250 can then be released out the sides and out the openings 30, 330 on the sidewalls 28, 328 of the implants 20, 320 where new growth is less urgent.

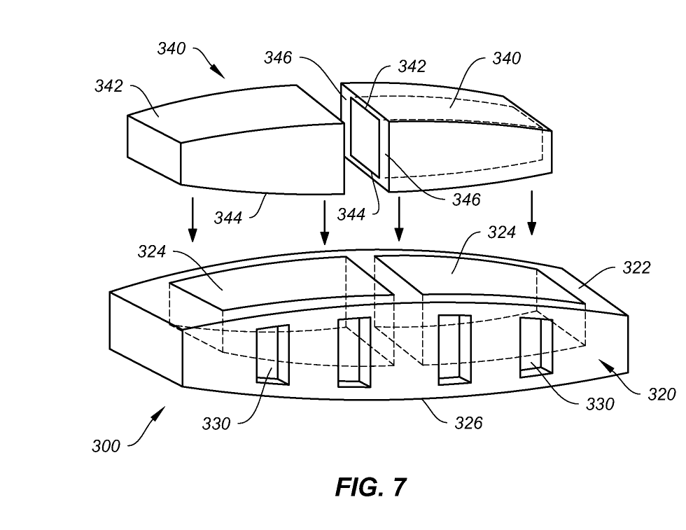

FIG. 7 illustrates an example of a system 300 in which the interbody implant 300 has more than one opening 324 for receiving the insert 340 of the present disclosure. As with the previous inserts, the inserts 340 may have superior and inferior surfaces 342, 344 that are thinner than the side surfaces 346 to release the contents of the insert 340, or agent, at the superior and inferior surfaces 342, 344 where contact with bleeding bone is optimal.

In one embodiment, the inserts 340 may have the same rates of release and control (i.e., same variable thickness at same location for each insert). However, it is also understood that the wall thicknesses or concentration of biological agent within the inserts 340 may also differ from one insert to another, such that the directionality of the new growth is not only relative to the spine, but also relative to the implant 320 itself. In other words, it is possible to control the degree of bone growth along the length of the implant 320 such that one end has a faster rate of activity than another end. This could be accomplished by providing an insert 340 containing more concentration of bioactive agent, or having thinner walls, at one opening 324, while using an insert 340 have less concentration of agent or having thicker walls, or both, in the other opening 324.

It is to be understood that, while the embodiments and drawings depict a spinal fusion implant, the biologically active insert of the present disclosure may also be used with other fusion implantable devices, including other bone fusion implants for other anatomical areas of the body. Further, more than one insert may be used to fill a single opening within the spinal fusion implant, such that a two or more inserts may be placed within an opening to fill up the space within the opening. In use, the biologically active component of the present disclosure may be inserted just prior to implantation of the fusion implant into the surgical site, or the fusion implant may be preloaded with the biologically active component and provided to the surgeon fully assembled for use.

The description of the invention is provided to enable any person skilled in the art to practice the various embodiments described herein. While the present invention has been particularly described with reference to the various figures and embodiments, it should be understood that these are for illustration purposes only and should not be taken as limiting the scope of the invention.

There may be many other ways to implement the invention. Various functions and elements described herein may be partitioned differently from those shown without departing from the spirit and scope of the invention. Various modifications to these embodiments will be readily apparent to those skilled in the art, and generic principles defined herein may be applied to other embodiments. Thus, many changes and modifications may be made to the invention, by one having ordinary skill in the art, without departing from the spirit and scope of the invention.

A reference to an element in the singular is not intended to mean “one and only one” unless specifically stated, but rather “one or more.” The term “some” refers to one or more. Underlined and/or italicized headings and subheadings are used for convenience only, do not limit the invention, and are not referred to in connection with the interpretation of the description of the invention. All structural and functional equivalents to the elements of the various embodiments of the invention described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

What is claimed is:

1. A biocompatible, bioresorbable insert comprising:
a polymeric shell containing a biologically active agent for facilitating bone fusion, the polymeric shell having varying thickness to provide directional and time-released control of the biologically active agent;
wherein the insert is configured to seat flush within an opening of a spinal fusion implant.

2. The insert of claim 1, wherein the polymeric shell comprises a biocompatible, bioabsorbable polymer or film-forming agent.

3. The insert of claim 2, wherein the polymer or film-forming agent comprises polycaprolactones (PCL), polyglycolic acid (PGA), poly-L-Lactic acid (PL-LA), polysulfones, polyolefins, polyvinyl alcohol (PVA), polyalkenoics, polyacrylic acids (PAA), PEG, PLGA, or polyesters.

4. The insert of claim 1, wherein the biologically active agent comprises a bone morphogenic protein, a peptide, a bone growth factor, a platelet derived growth factor, a vascular endothelial growth factor, an insulin derived growth factor, a keratinocyte derived growth factor, a fibroblast derived growth factor, a stem cell, bone marrow, or platelet rich plasma.

5. The insert of claim 1, wherein the spinal fusion implant comprises a posterior lumbar interbody fusion (PLIF) implant, transforaminal lumbar interbody fusion (TLIF) implant, anterior lumbar interbody fusion (ALIF) implant, or extreme lateral interbody fusion (XLIF) implant.

6. The insert of claim 1, wherein the polymeric shell comprises a superior wall, inferior wall, and sidewalls extending in between.

7. The insert of claim 6, wherein one of the superior or inferior walls is thinner than the sidewalls.

8. The insert of claim 1, wherein the biologically active agent is uniformly distributed throughout the insert.

9. The insert of claim 1, wherein the biologically active agent is non-uniformly distributed throughout the insert.

10. A spinal fusion implant system, comprising:
a spinal fusion implant having a superior surface, an inferior surface, sidewalls extending therebetween, and an opening extending from the superior surface through the inferior surface to accommodate bone growth therethrough; and
a biologically active component configured for insertion into the opening of the spinal fusion implant, the component including a polymeric shell containing a biologically active agent for facilitating bone fusion, the polymeric shell having varying thickness to provide directional and time-released control of the biologically active agent, wherein the component has a size and shape that complements the opening and is configured to seat flush within the opening of the spinal fusion implant.

11. The system of claim 10, wherein the polymeric shell comprises a biocompatible, bioabsorbable polymer or film-forming agent.

12. The system of claim 11, wherein the polymer or film-forming agent comprises polycaprolactones (PCL), polyglycolic acid (PGA), poly-L-Lactic acid (PL-LA), polysulfones, polyolefins, polyvinyl alcohol (PVA), polyalkenoics, polyacrylic acids (PAA), PEG, PLGA, or polyesters.

13. The system of claim 10, wherein the biologically active agent comprises a bone morphogenic protein, a peptide, a bone growth factor, a platelet derived growth factor, a vascular endothelial growth factor, an insulin derived growth factor, a keratinocyte derived growth factor, a fibroblast derived growth factor, a stem cell, bone marrow, or platelet rich plasma.

14. The system of claim 10, wherein the spinal fusion implant comprises a posterior lumbar interbody fusion (PLIF) implant, transforaminal lumbar interbody fusion (TLIF) implant, anterior lumbar interbody fusion (ALIF) implant, or extreme lateral interbody fusion (XLIF) implant.

15. The system of claim 10, wherein the opening is a central opening of the spinal fusion implant.

16. The system of claim 10, wherein the spinal fusion implant includes a plurality of openings, and the system further includes a plurality of components for insertion into the plurality of openings.

17. The insert of claim 10, wherein the polymeric shell comprises a superior wall, inferior wall, and sidewalls extending in between.

18. The insert of claim 17, wherein one of the superior or inferior walls is thinner than the sidewalls.

19. The insert of claim 10, wherein the biologically active agent is uniformly distributed throughout the component.

20. The insert of claim 10, wherein the biologically active agent is non-uniformly distributed throughout the component.
